(19) United States
(12) Patent Application Publication
(10) Pub. No.: US 2026/0256403 A1
(43) Pub. Date: Sep. 3, 2026

(54) PREMATURE HEARTBEAT ANALYSIS

(71) Applicant: Cardiac Pacemakers, Inc., St Paul, MN (US)

(72) Inventors: Riley Ellis, Rochester, MN (US); Jan Hagenbrock, Rochester, MN (US); David R. Engebretsen, Cannon Falls, MN (US); Timothy P. McClanahan, Rochester, MN (US); Javier G. Alonzo, San Francisco, CA (US)

(21) Appl. No.: 19/552,002

(22) Filed: Feb. 27, 2026

Related U.S. Application Data
(60) Provisional application No. 63/764,592, filed on Feb. 28, 2025.

(51) Int. Cl. A61B 5/344; A61B 5/339; A61B 5/352; G16H 40/67; G16H 50/30

(52) U.S. Cl. CPC A61B 5/344; A61B 5/339; A61B 5/352; G16H 40/67; G16H 50/30

(57) ABSTRACT

Systems and methods include approaches for calculating a relative timing value for respective beats associated with a rhythm classification, determining that respective relative timing values for a first set of the beats are within a first range of prematureness, and displaying time series cardiac data of the first set of the beats in a first window of a user interface.

### CROSS REFERENCE TO RELATED APPLICATION

This application claims priority to Provisional Application No. 63/764,592, filed February 28, 2025, which is herein incorporated by reference in its entirety.

### TECHNICAL FIELD

The present disclosure relates to devices, methods, and systems for monitoring, classifying, and displaying cardiac data.

### BACKGROUND

Monitoring devices for collecting biometric data are becoming increasingly common in diagnosing and treating medical conditions in patients. For example, mobile devices can be used to monitor cardiac data in a patient. This cardiac monitoring can empower physicians with valuable information regarding the occurrence of a variety of heart conditions and irregularities in patients. Cardiac monitoring can be used, for example, to identify abnormal cardiac rhythms, so that critical alerts can be provided to patients, physicians, or other care providers and patients can be treated.

### SUMMARY

In Example 1, a method includes calculating a relative timing value for respective beats associated with a rhythm classification, determining that respective relative timing values for a first set of the beats are within a first range of prematureness, and displaying time series cardiac data of the first set of the beats in a first window of a user interface.

In Example 2, the method of Example 1, further including determining that respective relative timing values for a second set of the beats are within a second range of prematureness that is different than the first range. The method further includes displaying time series cardiac data of the second set of the beats in a second window adjacent the first window.

In Example 3, the method of Example 2, further including displaying time series cardiac data of a third set of the beats in a third window adjacent the second window. The third set of the beats is associated with relative timing beats equal to or greater than an average heart rate of the beats associated with the rhythm classification.

In Example 4, the method of any of Examples 1–3, wherein the first range is based, at least in part, on a percentage difference below an average heart rate of the beats associated with the rhythm classification.

In Example 5, the method of any of Examples 1–4, wherein the respective relative timing values are based, at least in part, on respective RR intervals associated with the beats.

In Example 6, the method of Example 5, wherein the relative timing value for each of the beats is based on dividing the RR interval with the average heart rate of the beats associated with the rhythm classification.

In Example 7, the method of any of Examples 1–6, further including modifying, using the user interface, the first range by changing a minimum percentage or maximum percentage of the first range. The method further includes updating the time series cardiac data displayed in the first window.

In Example 8, the method of Example 7, wherein the minimum percentage or the maximum percentage are modified using a drop down menu or a textbox displayed on the user interface.

In Example 9, the method of any of Examples 1–8, wherein the time series data of the beats displayed in the first window is aligned with the beat’s respective R-wave peaks.

In Example 10, the method of any of Examples 1–9, further including selecting one of the beat’s time series data displayed in the first window and displaying, in another window, a strip of time series data occurring before and after the selected beat.

In Example 11, the method of any of Examples 1–10, further including modifying, using the user interface, a beat classification of the first set of the beats from a first beat classification to a second beat classification.

In Example 12, the method of Example 11, in response to the modifying, changing a rhythm classification of at least one cardiac event from a first rhythm classification to a second rhythm classification.

In Example 13, a computer program product comprising instructions to cause one or more processors to carry out the steps of the method of Examples 1–12.

In Example 14, a computer-readable medium having stored thereon the computer program product of Example 13.

In Example 15, a computer comprising the computer-readable medium of Example 14.

In Example 16, a method includes calculating a relative timing value for respective beats associated with a rhythm classification, determining that respective relative timing values for a first set of the beats are within a first range of prematureness, and displaying time series cardiac data of the first set of the beats in a first window of a user interface.

In Example 17, the method of Example 16, further including determining that respective relative timing values for a second set of the beats are within a second range of prematureness that is different than the first range. The method further includes displaying time series cardiac data of the second set of the beats in a second window adjacent the first window.

In Example 18, the method of Example 17, wherein the first set of beats and the second set of beats share a common beat classification.

In Example 19, the method of Example 18, wherein at least some the first set of beats and some of the second set of beats are associated with a common rhythm classification.

In Example 20, the method of Example 19, wherein at least some others of the first set of beats and some others of the second set of beats are associated with a different rhythm classification.

In Example 21, the method of Example 17, further including displaying time series cardiac data of a third set of the beats in a third window adjacent the second window. The third set of the beats is associated with relative timing beats equal to or greater than an average heart rate of the beats associated with the rhythm classification.

In Example 22, the method of Example 16, wherein the first range is based, at least in part, on a percentage difference below an average heart rate of the beats associated with the rhythm classification.

In Example 23, the method of Example 16, wherein the respective relative timing values are based, at least in part, on respective RR intervals associated with the beats.

In Example 24, the method of Example 23, wherein the relative timing value for each of the beats is based on dividing the RR interval with the average heart rate of the beats associated with the rhythm classification.

In Example 25, the method of Example 16, further including modifying, using the user interface, the first range by changing a minimum percentage or maximum percentage of the first range. The method further includes updating the time series cardiac data displayed in the first window.

In Example 26, the method of Example 25, wherein the minimum percentage or the maximum percentage are modified using a drop down menu or a textbox displayed on the user interface.

In Example 27, the method of Example 16, wherein the time series data of the beats displayed in the first window is aligned with the beat’s respective R-wave peaks.

In Example 28, the method of Example 16, further including selecting one of the beat’s time series data displayed in the first window and displaying, in another window, a strip of time series data occurring before and after the selected beat.

In Example 29, the method of Example 16, further including modifying, using the user interface, a beat classification of the first set of the beats from a first beat classification to a second beat classification.

In Example 30, the method of Example 29, in response to the modifying, changing a rhythm classification of at least one cardiac event from a first rhythm classification to a second rhythm classification.

In Example 31, a system includes a remote computing system with a user interface, one or more processors, and a computer-readable medium having a set of computer-executable instructions. When executed by the one or more processors, the computer-executable instructions are configured to cause the remote computing system to: calculate a relative timing value for respective beats associated with a rhythm classification, determine that respective relative timing values for a first set of the beats are within a first range of prematureness, and display time series cardiac data of the first set of the beats in a first window in the user interface.

In Example 32, the system of Example 31, wherein the first range is based, at least in part, on a percentage difference below an average heart rate of the beats associated with the rhythm classification.

In Example 33, the system of Example 31, wherein the respective relative timing values are based, at least in part, on respective RR intervals associated with the beats.

In Example 34, the system of Example 33, wherein the relative timing value for each of the beats is based on dividing the RR interval with the average heart rate of the beats associated with the rhythm classification.

In Example 35, the system of Example 31, wherein the time series data of the beats displayed in the first window is aligned with the beat’s respective R-wave peaks.

In Examiner 36, the method or system of Examples 1–35, wherein the time series cardiac data is electrocardiogram data.

While multiple instances are disclosed, still other instances of the present invention will become apparent to those skilled in the art from the following detailed description, which shows and describes illustrative instances of the invention. Accordingly, the drawings and detailed description are to be regarded as illustrative in nature and not restrictive.

### BRIEF DESCRIPTION OF THE DRAWINGS

FIG. 1 shows a cardiac monitoring system, in accordance with certain instances of the present disclosure.

FIG. 2 shows a server, a remote computer, and a user interface, in accordance with certain instances of the present disclosure.

FIG. 3 shows an example of beats that have been grouped together, in accordance with certain instances of the present disclosure.

FIG. 4 shows a window of a user interface, in accordance with certain instances of the present disclosure.

FIGS. 5 and 6 show various portions of a user interface, in accordance with certain instances of the present disclosure.

FIG. 7 shows a block diagram depicting an illustrative method, in accordance with certain instances of the disclosure.

FIG. 8 is a block diagram depicting an illustrative computing device, in accordance with instances of the disclosure.

While the invention is amenable to various modifications and alternative forms, specific instances have been shown by way of example in the drawings and are described in detail below. The intention, however, is not to limit the invention to the particular instances described. On the contrary, the invention is intended to cover all modifications, equivalents, and alternatives falling within the scope of the invention as defined by the appended claims.

### DETAILED DESCRIPTION

Cardiac data such as electrocardiogram (ECG) data of a patient can be used to analyze and diagnose a patient’s cardiac activity and recommend treatments. To collect ECG data, one or more monitoring devices (e.g., sensors) can be coupled to the patient such that the monitoring devices sense and record the ECG data. The ECG data can be processed using one or more machine learning models, which output data such as heartbeat classifications, rhythm (and/or event) classifications, heart rates, etc.

The machine learning model(s) can associate individual beats with a rhythm, and each rhythm predicted by the machine learning model(s) can be assigned a classification. The beat classifications and the rhythm classifications can be further processed and analyzed by a human using a user interface. For example, the user interface can be used to alter the original beat classifications and/or the original rhythm classifications generated by the machine learning model(s).

One reason why original beat classifications may be altered is because certain beats associated within a rhythm may be premature relative to other beats associated with that rhythm. For example, the premature beats may have an RR interval that is shorter relative to the RR intervals or the average heart rates of other beats associated with the same type of rhythm. Misclassification of beats may occur, for example, when beats have similar shapes (e.g., morphologies) but different heart rates. For example, normal beats and supraventricular beats typically have similar shapes, but supraventricular beats are associated with faster heart rates than normal beats. Certain instances of the present disclosure involve approaches for identifying and displaying premature beats.

### CARDIAC MONITORING SYSTEM

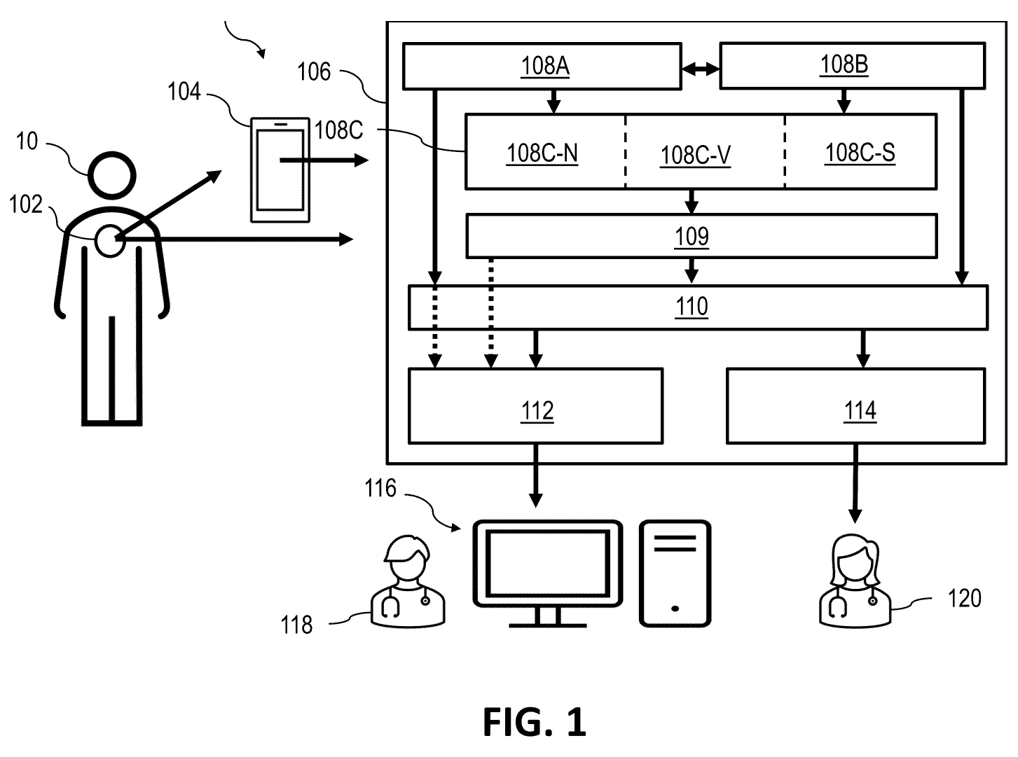

FIG. 1 illustrates a patient 10 and an example system 100. The system 100 includes a monitor 102 attached to the patient 10 or implanted in the patient 10 (e.g., pacemaker, ICD, CRT, or ICM) to detect cardiac activity of the patient 10. The monitor 102 may produce electric signals that represent the cardiac activity in the patient 10. For example, the monitor 102 may detect the patient’s heart beating (e.g., using infrared sensors, electrodes, heart sounds) and convert the detected heartbeat into electric signals representing ECG data. In certain instances, the monitor 102 stores the ECG data of a patient study (e.g., one or more days of ECG data), after which the ECG data is transmitted to another device or system such as a server. Additionally or alternatively, the monitor 102 transmits the ECG data to a mobile device 104 (e.g., a mobile phone). In such instances, the mobile device 104 can include a program (e.g., mobile phone application) that receives, processes, and analyzes the ECG data. For example, the program may analyze the ECG data and detect or flag cardiac events (e.g., periods of irregular cardiac activity) contained within the ECG data.

The mobile device 104 can periodically transmit chunks of the ECG data to another device or system such as a server, which can process, append together, and archive the chunks of the ECG data and metadata (e.g., time, duration, detected/flagged cardiac events) associated with the chunks of ECG data. In certain instances, the monitor 102 may be programmed to transmit the ECG data directly to the other device or system without utilizing the mobile device 104. Also, the monitor 102 and/or the mobile device 104 includes a button or touch-screen icon that allows the patient 10 to initiate an event. Such an indication can be recorded and communicated to the other device or system. In other instances involving multi-day studies, the ECG data and associated metadata are transmitted in larger chunks (e.g., an entire study’s worth of ECG data).

### CARDIAC EVENT SERVER

The ECG data (and associated metadata, if any) is transmitted to and stored by a cardiac event server 106 (hereinafter “the server 106” for brevity). The server 106 can include multiple models, platforms, layers, or modules that work together to process and analyze the ECG data such that cardiac events can be detected, filtered, prioritized, and ultimately reported to a patient’s physician for analysis and treatment. In the example of FIG. 1, the server 106 includes one or more machine learning models 108A, 108B, and 108C, a clustering algorithm module 109, a cardiac event router 110, a report platform 112, and a notification platform 114. Although only one server 106 is shown in FIG. 1, the server 106 can include multiple separate physical servers, and the various models/platforms/modules/layers can be distributed among the multiple servers. Each of the models/platforms/modules/layers can represent separate programs, applications, and/or blocks of code where the output of one of the models/platforms/ modules/layers is an input to another of the models/platforms/modules/layers. Each of the models/platforms/modules/layers can use application programming interfaces to communicate between or among the other models/platforms/modules/layers as well as systems and devices external to the server 106.

In certain instances, once the ECG data is processed by the machine learning models 108A–C and the clustering algorithm module 109, the ECG data (and associated metadata) is made available for the report platform 112. As will be described in more detail below, the report platform 112 can be accessed by a remote computer 116 (e.g., client device such as a laptop, mobile phone, desktop computer, and the like) by a user at a clinic or lab 118. In other instances, the cardiac event router 110 is used to determine what platform further processes the ECG data based on the classification associated with the cardiac event. For example, if the identified cardiac event is critical or severe, the cardiac event router 110 can flag or send the ECG data, etc., to the notification platform 114. The notification platform 114 can be programmed to send notifications (along with relevant ECG data and associated metadata) immediately to the patient’s physician/care group remote computer 116 and/or to the patient 10 (e.g., to their computer system, e-mail, mobile phone application).

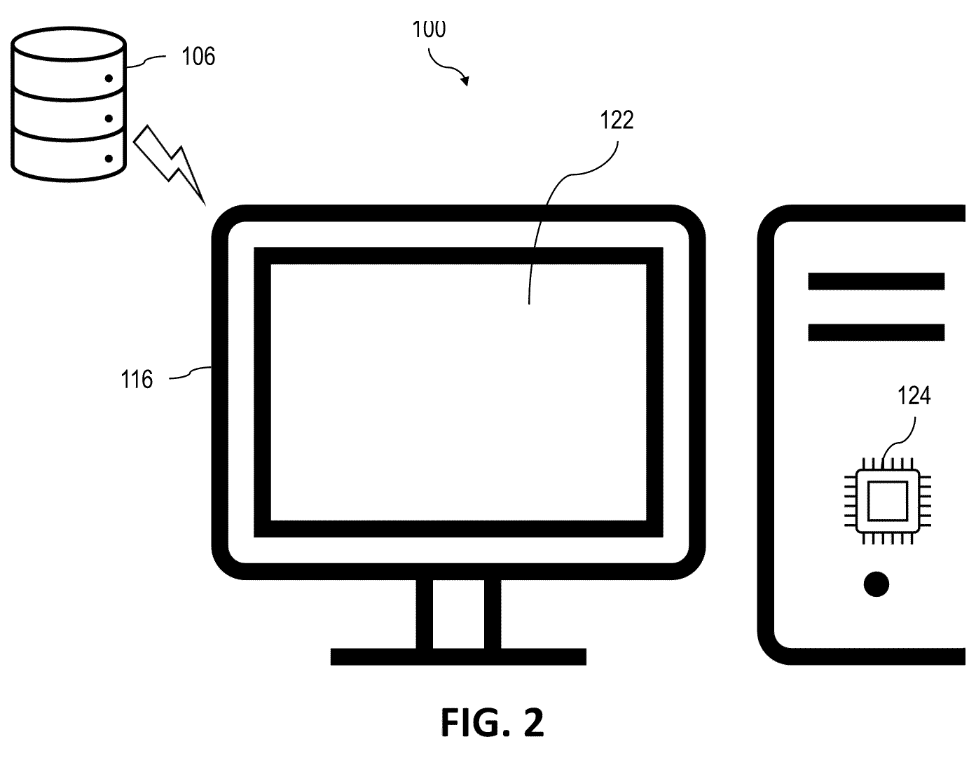

FIG. 2 shows the server 106 communicatively coupled (e.g., via a network) to the remote computer 116. In the example of FIG. 2, the remote computer 116 includes a monitor showing a user interface 122 (hereinafter “the UI 122” for brevity) that displays features of the report platform 112 hosted by the server 106. The UI 122 includes multiple pages or screens for tracking and facilitating analysis of patient ECG data.

In certain instances, the report platform 112 is a software-as-a-service (SaaS) platform hosted by the server 106. To access the report platform 112, a user (e.g., a technician) interacts with the UI 122 to log into the report platform 112 via a web browser such that the user can use and interact with the report platform 112.

### MACHINE LEARNING MODELS

Referring back to FIG. 1, the server 106 applies the one or more machine learning models 108A–C to the ECG data to analyze and classify the beats and cardiac activity of the patient 10.

The first and second machine learning models 108A and 108B are programmed to—among other things—compare the ECG data to labeled ECG data to determine which labeled ECG data the ECG data most closely resembles. The labeled ECG data may identify a particular cardiac event—including but not limited to ventricular tachycardia, bradycardia, atrial fibrillation, pause, normal sinus rhythm, or artifact/noise—as well as particular beat classifications—including but not limited to ventricular, normal, or supraventricular beats. In addition to identifying beat classifications and event classifications (and generating associated metadata), the first and second machine learning models 108A and 108B can determine and generate metadata regarding heart rates, duration, and beat counts of the patient 10 based on the ECG data. As specific examples, the first and/or the second machine learning models 108A and 108B can identify the beginning, center, and end of individual beats (e.g., individual T-waves) such that individual beats can be extracted from the ECG data. Each individual beat can be assigned a value (e.g., a unique identifier) such that individual beats can be identified and associated with metadata throughout processing and analyzing the ECG data.

The ECG data (e.g., ECG data associated with individual beats) as well as certain outputs of the first and second machine learning models 108A and 108B can be inputted to the third machine learning model 108C. Although two machine learning models are shown and described, a single machine learning model could be used to generate the metadata described herein, or additional machine learning models could be used.

The first and second machine learning models 108A and 108B can include the neural networks described in U.S. Pat. App. No. 16/695,534, which is hereby incorporated by reference in its entirety. The first neural network can be a deep convolutional neural network and the second neural network is a deep fully-connected neural network—although other types and combinations of machine learning models can be implemented. The first machine learning model 108A receives one or more sets of beats (e.g., beat trains with 3–10 beats) which are processed through a series of layers in the deep convolutional neural network. The series of layers can include a convolution layer to perform convolution on time series data in the beat trains, a batch normalization layer to normalize the output from the convolution layer (e.g., centering the results around an origin), and a non-linear activation function layer to receive the normalized values from the batch normalization layer. The beat trains then pass through a repeating set of layers such as another convolution layer, a batch normalization layer, a non-linear activation function layer. This set of layers can be repeated multiple times.

The second machine learning model 108B receives RR-interval data (e.g., time intervals between adjacent beats) and processes the RR-interval data through a series of layers: a fully connected layer, a non-linear activation function layer, another fully connected layer, another non-linear activation function layer, and a regularization layer. The output from the two paths is then provided to the fully connected layer. The resulting values are passed through a fully connected layer and a softmax layer to produce probability distributions for the classes of beats.

The third machine learning model 108C (e.g., one or more trained encoder machine learning models) is programmed to generate latent space representations of the ECG data such that the ECG data is represented by fewer datapoints than the original ECG data. The latent space representations can be used as an approximation of the original raw ECG data for each beat. Although the inputs to the third machine learning model 108C are described as (1) the ECG data such as sets of individual T-waves and (2) certain outputs of the first and second machine learning models 108A and 108B, the third machine learning model 108C could be programmed to generate the latent space representations without requiring input from the first and/or second machine learning models 108A, 108B.

In certain instances, instead of a single third machine learning model 108C, the server 106 includes a separate machine learning model for each type of beat classification (e.g., normal beats, ventricular beats, and supraventricular beats). For example, as shown in FIG. 1, the server 106 may include three third machine learning models (108C-N, 108C-V, and 108C-S) instead of a single third machine learning model. In certain instances, beats that were not initially classified (e.g., unclassified beats) can be processed either by a different third machine learning model or can skip the step of generating latent space representations and being clustered with similar shaped beats.

In the example of FIG. 1, one machine learning model 108C-N is used for beats classified as normal beats, another machine learning model 108C-V is used for beats classified as ventricular beats, and another machine learning model 108C-S is used for beats classified as supraventricular beats. As such, only ECG data (e.g., T-waves) of beats initially classified as normal beats by the first and/or second machine learning models 108A, 108B—as well as metadata generated by such machine learning models—are inputted to the machine learning model 108C-N, and so on. It has been found that using machine learning models trained to focus on analyzing only certain types of beats can improve performance of the third machine learning models compared to using a single third machine learning model. Further, processing the ECG data in parallel using three machine learning models can decrease the time needed to generate the latent space representations. In certain instances, a single study may contain hundreds of thousands to millions of individual beats.

Each third machine learning model (108C-N, 108C-V, 108C-S) receives ECG data associated with individual beats (e.g., an individual clip of ECG data for each beat) and generates latent space representations of such ECG data. For example, each individual beat is processed by one of the third machine learning models—depending on each individual beat’s classification—such that the ECG data is distilled down to (or represented by) a small number of individual data points. Raw ECG data of an individual beat can include 500 or so datapoints, and each third machine learning model can distill the ECG data for a given beat into 4–16 datapoints. Put another way, each third machine learning model can generate latent space representations comprising 4–16 datapoints for a given beat. This range has been found to balance accuracy of beat representation and effectiveness of clustering (described further below). In certain instances, the latent space representations comprise 7, 8, or 9 (e.g., 7–9) datapoints for a given beat. The latent space representations comprise 1–2% of datapoints compared to the raw ECG data for each beat. Each latent space can be represented by a vector (e.g., a latent vector).

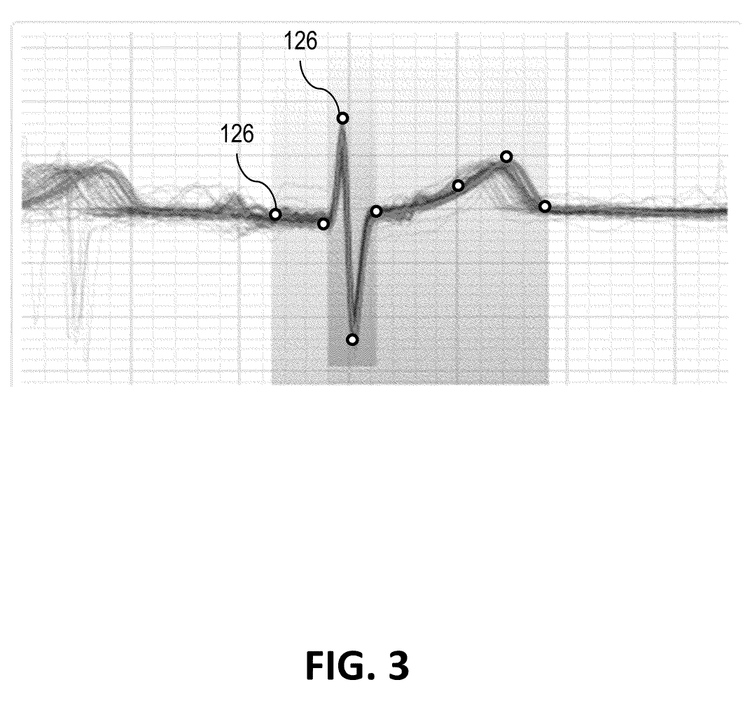

The resulting datapoints are representations of an amplitude of the ECG signal at different relative points in time. These limited datapoints are datapoints that the trained machine learning models generate such that different beat shapes can be identified and similar shaped beats can be grouped together. Put another way, these datapoints may be those that are the most likely to be helpful in distinguishing among beat shapes. The third machine learning models can leave out representations of datapoints that are less likely to help distinguish among individual beats. FIG. 3 shows an example set of beats that have been grouped or clustered together and also shows non-limiting examples of points 126 within a beat’s ECG signal that may be useful for distinguishing among beat shapes. For example, the points 126 can be located at the beginning and end of each beat, apexes (e.g., QRS peaks), nadirs, etc.

In the example of FIG. 1, the third machine learning models (108C-N, 108C-V, 108C-S) generate respective separate latent space representations for sets of beats initially classified as normal beats, ventricular beats, and supraventricular beats. In certain instances, beats that could not be initially classified (or ECG data containing artifacts due to noise) are not processed by any of the third machine learning models. Such beats can be labeled as unclassified beats.

The output(s) of the third machine learning model(s) 108C are processed by a clustering algorithm module 109. The clustering algorithm module 109 receives the latent space representations of individual beats and is programmed to associate similar shaped beats into different groups. FIG. 3 shows an example set of beats that have been grouped or clustered together. As shown in FIG. 3, ECG waveforms of individual beats (e.g., T-waves) are superimposed on each other. Each cluster or group can include hundreds or thousands of beats that have been grouped together by the clustering algorithm module 109. As can be seen, the beats all have a similar profile relative to each other. Each beat is aligned with the other beats to have respective QRS peaks centered on the graph.

In certain instances, the clustering algorithm module 109 is programmed to apply a clustering algorithm such as the k-means clustering algorithm or a derivation or variation thereof to the latent space representations. In certain instances, the same clustering algorithm module 109 and the same algorithm is used to process the latent space representations of each of the third machine learning models (108C-N, 108C-V, 108C-S). In certain instances, the output of the clustering algorithm module 109 includes assigning a value (e.g., an identifier such as a number) to each beat that is indicative of the group selected by the clustering algorithm module 109. For example, if the clustering algorithm module 109 clusters the beats into eight different groups, then all beats selected to be in the first group may be assigned a value of “1” and all beats selected to be in the second group may be assigned a value of “2” and so on. Other types of values can be used. These group values can be added to the metadata associated with each beat.

Accessing and displaying days of ECG data can be an inefficient use of computing resources, network bandwidth resources, and human resources. To help address these resource challenges, the remote computer 116 and the server 106 can communicate with each other (e.g., via commands/requests and responses) to prioritize when and what ECG data and metadata are accessible to users at the remote computer 116. The remote computer 116 can initiate commands/requests that are transmitted to the server 106, and the server 106 transmits ECG data and metadata in response to the commands/requests. For example, in some instances, the remote computer 116 receives executable code (e.g., JavaScript code) as part of an initial batch of files from the server 106, and the executable code includes code for requesting and prioritizing retrieval of data from the server 106.

The data can be downloaded in response to the remote computer 116 sending commands or requests to the server 106 for particular sets of data. For example, before raw ECG data is downloaded to the remote computer 116, the remote computer 116 can send a command or request to the server 106 for certain metadata. This metadata can include non-ECG patient data (e.g., name, physician) and pointers to ECG data and associated metadata. In certain instances, the pointers are used by the remote computer 116 to request specific strips of ECG data (e.g., specific time periods of ECG data) stored at the server 106 be downloaded to the remote computer 116. As such, to download particular strips of ECG data, the remote computer 116 can utilize the pointers to request the strips from the server 106.

The initial batch of metadata can be downloaded in a first data payload. Additionally or alternatively, the metadata initially downloaded can include beat data (e.g., classifications, locations in time, associated strip of ECG data) and cardiac event data (e.g., classifications, locations in time, associated strip of ECG data). This batch of metadata can be downloaded in a second data payload. Also, in certain instances, once a patient study session is selected, the remote computer 116 receives the executable code as described above. In other instances, the executable code is downloaded to the remote computer 116 when a user initially accesses the report platform 112 and before a patient study session is selected.

As strips of ECG data (or portions thereof) have been downloaded to the remote computer 116, the ECG and associated metadata can be displayed on the UI 122. For example, plots of ECG data can be displayed in one or more windows of the UI 122.

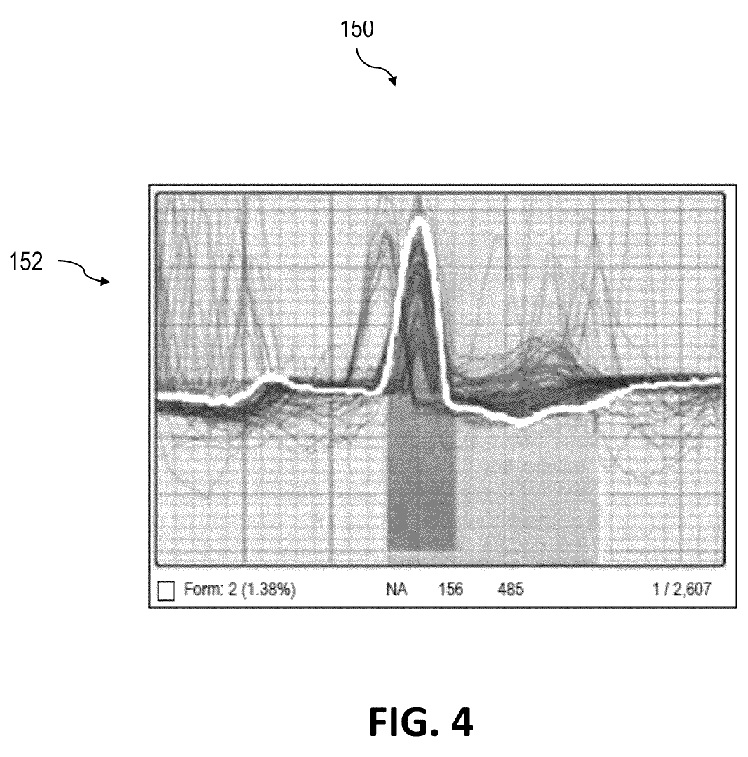

FIG. 4 shows a portion of a user interface (UI) 150. In FIG. 4, the UI 150 is shown displaying together plots of time series cardiac data (e.g., ECG data) in a window 152. The window 152 can display multiple plots of individual beats (e.g., beat-sized strips of time series cardiac data) that have the same beat classification (e.g., normal beats, ventricular beats, supraventricular beats, and unknown beats as determined by the clustering algorithm module 109) and/or that are associated with the same type of rhythm (e.g., the same type of cardiac event such as atrial fibrillation, bradycardia, tachycardia, and the like).

As shown, window 152 includes multiple plots that are superimposed on each other. The window 152 can display plots of hundreds or more of beats that have been grouped together (e.g., by the autoencoder DNN). Each beat can be aligned to have its QRS peak centered along the same vertical axis so that the beats can be visually compared to each other.

### PREMATURE HEARTBEAT VIEW

As noted herein, certain beats associated with a rhythm may be premature relative to other beats associated with that rhythm. Premature beats may be inaccurately classified as a normal beat instead of an abnormal beat (e.g., a ventricular beat, a supraventricular beat). For example, normal beats and supraventricular beats typically have similar shapes or morphologies, but supraventricular beats are associated with faster heart rates than normal beats.

It can be a power- and time-intensive challenge to not only identify premature beats within a study but also to correct misclassified beats. For example, a study such as a Holter study may include thousands or hundreds of thousands of beats associated within a given type of rhythm (e.g., tachycardia-type rhythms, bradycardia-type rhythms). Various approaches described herein can be used to identify premature beats and correct misclassified beats en masse.

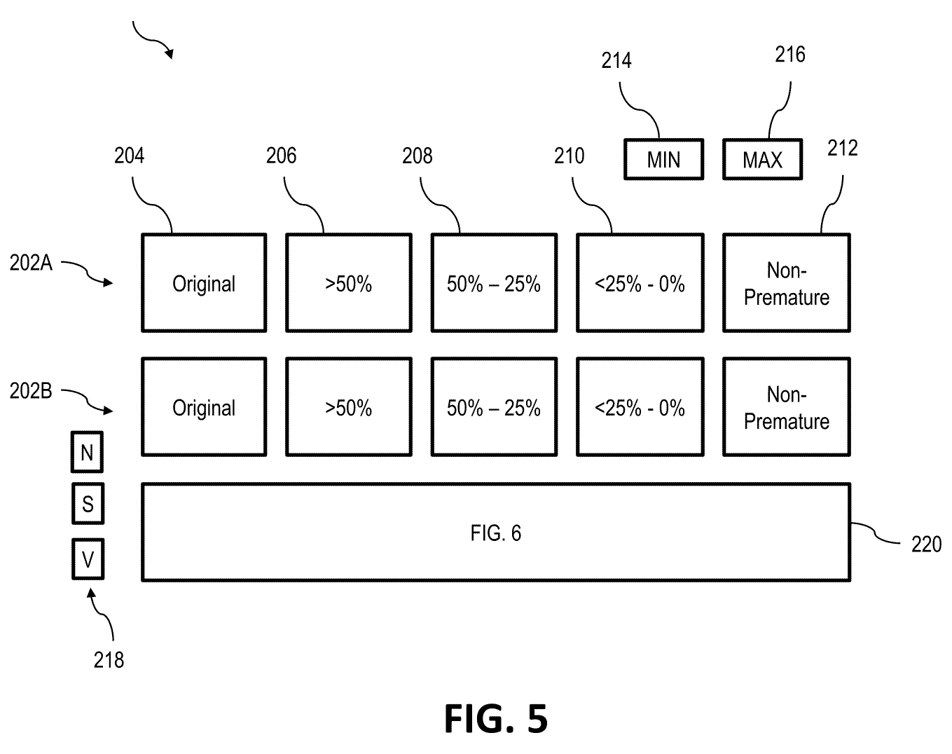

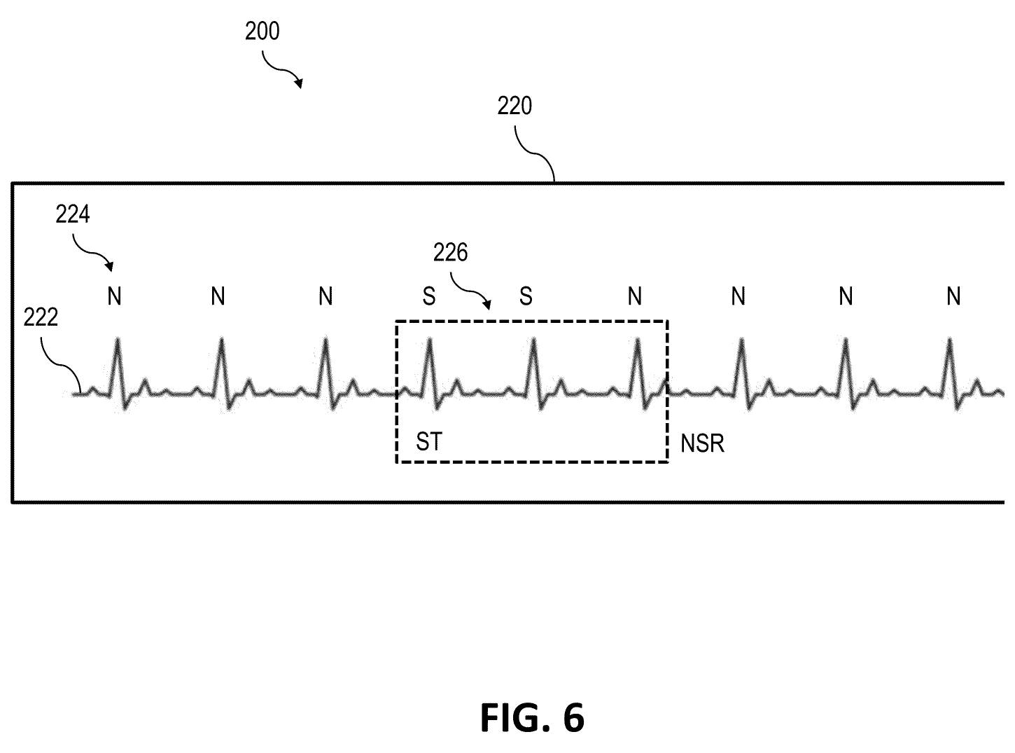

FIGS. 5 and 6 show schematics of portions of a user interface (UI) 200.

FIG. 5 shows the UI 200 with one or more rows 202A, 202B of windows 204–212. Each of the windows 204–212 can display multiple plots of individual beats such as the window 152 shown in FIG. 4. For example, each window 204–212 can include multiple plots of ECG data that are superimposed on each other and that are centered along the same vertical axis so that the beats can be visually compared to each other.

Each window 204–212 can display different groupings of beats. For example, within the first row 202A, the UI 200 can include one window 204 for displaying the original set of beats that are associated with a specific type of rhythm. In some instances, within the first row 202A, the beats displayed are not only those associated with a specific type of rhythm but also those beats associated with a specific beat classification. In other instances, the beats displayed within the first row 202A all have the same current beat classification but can be associated with different rhythm classifications. The remaining windows 206–212 in the first row 202A can display the beats with respective different ranges of prematureness relative to the other beats. A user can select a different type of beats to alter which type of beats are displayed in the windows of the first row 202A.

The windows in the second row 202B can display beats in similar groupings. However, the windows in the second row 202B can display beats associated with a different type of rhythm and/or a different type of beat classification.

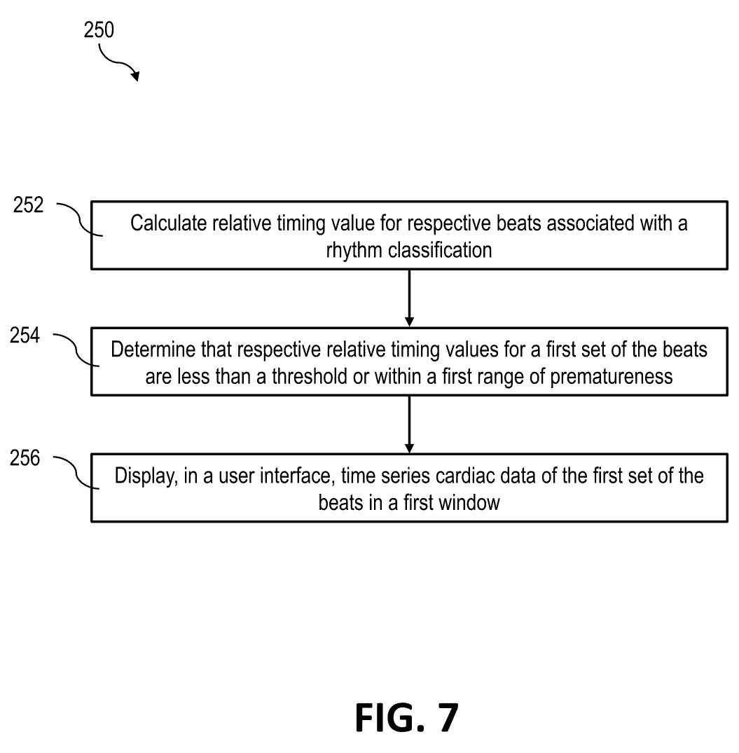

FIG. 7 outlines a method 250 for determining and displaying beats with different ranges of relative prematureness. The method 300 includes calculating a relative timing value for respective beats associated with a rhythm classification (block 252 in FIG. 7). Put another way, a relative timing value can be calculated for each beat associated with a specific rhythm classification or sub-rhythm classification (e.g., different tachycardia-type rhythms, different bradycardia-type rhythms).

In certain instances, the relative timing value is based on an average heart rate of the beats associated with the specific rhythm classification. As one specific example, the relative timing value for each beat can be based on the RR interval of the beat divided by the average heart rate of the beats associated with the specific rhythm classification. In certain instances, the average heart rate is the average of a respective beat’s instantaneous heart rate, the overall average RR interval within the specific rhythm classification, or the overall length of the rhythm divided by the number of beats within the rhythm. In other instances, the relative timing value is based on comparing a given beat’s instantaneous heart rate to the instantaneous heart rates of the beats surrounding it. In other instances, the relative timing value is based on a rhythm’s average heart rate and the instantaneous heart rates of the surrounding beats.

The relative timing value for each beat can be each beat’s percentage difference between the average heart rate of the beats associated with the rhythm classification. Put another way, the relative timing value can be expressed in terms of a percentage difference from the average heart rate within the specific rhythm classification. For example, an individual beat’s relative timing value may indicate that a given beat is a certain percentage (e.g., 0%<) below an average heart rate.

The relative timing value can be used to identify premature beats. For example, premature beats can be grouped together by determining that respective relative timing values for a first set of the beats are less than a threshold or within a first range of prematureness (block 254 in FIG. 7). In certain instances, the threshold is the average heart rate of beats associated with the specific rhythm classification. In other instances, the first range of prematureness is a range below the average heart rate of beats associated with the specific rhythm classification.

Once certain premature beats are grouped together, the beats can be displayed in a window of the UI 200. For example, time series cardiac data of the first set of the beats can be displayed in a first window (block 256 in FIG. 7).

Referring back to FIG. 5, the windows 206–212 can display time series cardiac data of different groupings of beats. The windows 206–210 can display groupings of beats with different ranges of prematureness for the rhythm classification.

The window 206 can display time series cardiac data of beats that have a relative timing value that indicates that the beat is greater than 50% premature compared to other beats associated with the rhythm classification (e.g., relative to an average heart rate of the rhythm classification).

The window 208 adjacent to the window 206 can display time series cardiac data of beats that have a relative timing value that indicates that the beat is 50% or less and greater than 25% premature compared to other beats associated with the rhythm classification.

The window 210 adjacent to the window 208 can display time series cardiac data of beats that have a relative timing value that indicates that the beat is less than 25% premature compared to other beats associated with the rhythm classification.

The window 212 adjacent to the window 210 can display time series cardiac data of beats that are not premature based on the relative timing value calculated for each beat.

In certain instances, each window 206–210 can initially default to a predetermined range of prematureness. However, the UI 200 can include one or more features 214, 216 that allow a user to adjust one or more end points of the ranges of the windows 206–210. For example, one of the features 214 on the UI 200 can include an editable text box, a drop down menu with preselected percentages, a button, a dial, etc., that can be used to adjust the minimum of a range. As another example, one of the features 216 on the UI 200 can include an editable text box, a drop down menu with preselected percentages, a button, a dial, etc., that can be used to adjust the maximum of a range.

Once the minimum and/or the maximum of a given range of prematureness is modified using the UI 200, the associated window can update which beats are displayed within the window.

Using the windows 206–210 and features 214, 216; a user can use the UI 200 to efficiently identify groups of premature beats to display and evaluate.

In addition to displaying different range-based groupings of premature beats, the windows 206–210 can be used to modify beat classifications. For example, for beats associated with one of the windows 206–210, a user can select all such beats and change their associated beat classification. To assist with this reclassification, the UI 200 can include features 218 such as buttons that represent different beat classifications (e.g., “N” for normal, “V” for ventricular, and “S” for supraventricular), and the desired feature 218 can be selected to cause the beat classifications for the selected beats to change. When the beat classification is changed, the metadata associated with the selected beats can be modified to represent the updated beat classification. Using the UI 200, metadata for hundreds to thousands to hundreds of thousands (or millions, for long studies) of beats can be updated en masse.

Changing the classification of one or more beats can lead to automatically reclassifying rhythms containing the changed beats—instead of a user manually analyzing and reclassifying each rhythm. For example, when premature beats originally classified as normal beats are changed to ventricular beats, the system may recalculate rhythm classifications and determine that time periods that were originally determined to contain normal sinus rhythms should be reclassified as containing ventricular-type rhythm events.

The windows 206–210 can also be used to select an individual beat displayed within one of the windows 206–210 to display time series cardiac data surrounding the selected beat. For example, once a user selects an individual beat’s time series data displayed in one of the windows 206–210, a longer strip of time series data occurring before and after the selected beat can be displayed in another window 220. To select a beat, a user can use a displayed cursor (controlled by a mouse, keyboard, etc.) to select a plot that is displayed in one of the windows 206–210. Once a plot is selected, the selected plot(s) can be displayed in a different color, line weight, and/or line type than the rest of the plots in the window.

FIG. 6 shows an example of the type of data and information (e.g., metadata) that can be displayed in the window 220 of FIG. 5. The window 220 can display a plot 222 of time series cardiac data. The window 220 can also display heartbeat classification indicators 224 for each beat that is displayed within the time series cardiac data. The heartbeat classification indicators 224 can represent the beat classifications initially generated by machine learning models. The heartbeat classification indicators 224 can include a single letter such as “S” for representing supraventricular beats, “V” for representing ventricular beats, and “N” for representing normal beats. In certain instances, the heartbeat classification indicators 224 are positioned adjacent to the time series cardiac data such as immediately above a peak of each beat (e.g., immediately above the peak of the “R” wave of the QRS interval).

As another example of displayed metadata, the window 220 can display rhythm classification indicators 226 adjacent to the time series cardiac data. The rhythm classification indicators 226 can represent the rhythm classifications initially generated by machine learning models. The rhythm classifications can be classifications associated with multiple beats — as opposed to beat classifications, which can be associated with an individual beat. The rhythm classification indicators 226 can include one or more letters representing a specific type of rhythm such as “ST” for supraventricular tachycardia, “NSR” for normal sinus rhythm, “P” for pause, etc. In addition, the rhythm classification indicators 226 can include a ribbon or box, which is represented by dotted lines in FIG. 6. In certain instances, the ribbon or box is only used to indicate abnormal rhythms (e.g., rhythms other than NSR). In certain instances, the rhythm classification indicators 226 are aligned with a starting time (e.g., onset) and ending time of a given cardiac event.

Once the user or users are satisfied with the analysis of the study, a final report can be generated and sent to the patient’s physician. In certain instances, once the report is built and complete, the remote computer 116 can send any changes to the metadata (e.g., the beat classifications, the rhythm classifications, start times, end times) to the server 106 and its database. The server 106 can then replace the metadata initially created by the machine learning model (and saved to the database) with the metadata generated by the remote computer while the user was reviewing and editing the metadata. As such, if the ECG data and metadata need to be accessed again, the server’s database has the most recent version of the metadata. Further, machine learning model may be further trained on the metadata generated by the user at the remote computer.

### COMPUTING DEVICES AND SYSTEMS

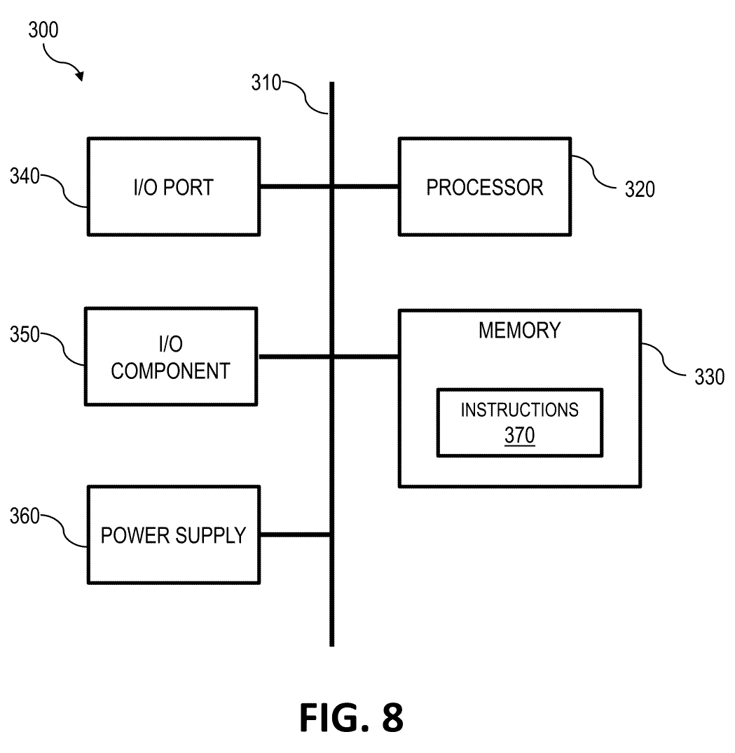

FIG. 8 is a block diagram depicting an illustrative computing device 300, in accordance with instances of the disclosure. The computing device 300 may include any type of computing device suitable for implementing aspects of instances of the disclosed subject matter. Examples of computing devices include specialized computing devices or general-purpose computing devices such as workstations, servers, laptops, desktops, tablet computers, hand-held devices, smartphones, general-purpose graphics processing units (GPGPUs), and the like. Each of the various components shown and described in the Figures can contain their own dedicated set of computing device components shown in FIG. 8 and described below. For example, the monitor 102, the mobile device 104, the server 106, and the remote computer 116 can each include their own set of components shown in FIG. 8 and described below.

In instances, the computing device 300 includes a bus 310 that, directly and/or indirectly, couples one or more of the following devices: a processor 320, a memory 330, an input/output (I/O) port 340, an I/O component 350, and a power supply 360. Any number of additional components, different components, and/or combinations of components may also be included in the computing device 300.

The bus 310 represents what may be one or more busses (such as, for example, an address bus, data bus, or combination thereof). Similarly, in instances, the computing device 300 may include a number of processors 320, a number of memory components 330, a number of I/O ports 340, a number of I/O components 350, and/or a number of power supplies 360. Additionally, any number of these components, or combinations thereof, may be distributed and/or duplicated across a number of computing devices.

In instances, the memory 330 includes computer-readable media in the form of volatile and/or nonvolatile memory and may be removable, nonremovable, or a combination thereof. Media examples include random access memory (RAM); read only memory (ROM); electronically erasable programmable read only memory (EEPROM); flash memory; optical or holographic media; magnetic cassettes, magnetic tape, magnetic disk storage or other magnetic storage devices; data transmissions; and/or any other medium that can be used to store information and can be accessed by a computing device. In instances, the memory 330 stores computer-executable instructions 370 for causing the processor 320 to implement aspects of instances of components discussed herein and/or to perform aspects of instances of methods and procedures discussed herein. The memory 330 can comprise a non-transitory computer readable medium storing the computer-executable instructions 370.

The computer-executable instructions 370 may include, for example, computer code, machine-useable instructions, and the like such as, for example, program components capable of being executed by one or more processors 320 (e.g., microprocessors) associated with the computing device 300. Program components may be programmed using any number of different programming environments, including various languages, development kits, frameworks, and/or the like. Some or all of the functionality contemplated herein may also, or alternatively, be implemented in hardware and/or firmware.

According to instances, for example, the instructions 370 may be configured to be executed by the processor 320 and, upon execution, to cause the processor 320 to perform certain processes. In certain instances, the processor 320, memory 330, and instructions 370 are part of a controller such as an application specific integrated circuit (ASIC), field-programmable gate array (FPGA), and/or the like. Such devices can be used to carry out the functions and steps described herein.

The I/O component 350 may include a presentation component configured to present information to a user such as, for example, a display device, a speaker, a printing device, and/or the like, and/or an input component such as, for example, a microphone, a joystick, a satellite dish, a scanner, a printer, a wireless device, a keyboard, a pen, a voice input device, a touch input device, a touch-screen device, an interactive display device, a mouse, and/or the like.

The devices and systems described herein can be communicatively coupled via a network, which may include a local area network (LAN), a wide area network (WAN), a cellular data network, via the internet using an internet service provider, and the like.

Aspects of the present disclosure are described with reference to flowchart illustrations and/or block diagrams of methods, devices, systems and computer program products. It will be understood that each block of the flowchart illustrations and/or block diagrams, and combinations of blocks in the flowchart illustrations and/or block diagrams, can be implemented by computer program instructions.

Various modifications and additions can be made to the exemplary embodiments discussed without departing from the scope of the present invention. For example, the scope of this invention includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A method comprising:
calculating a relative timing value for respective beats associated with a rhythm classification;
determining that respective relative timing values for a first set of the beats are within a first range of prematureness; and
displaying, in a user interface, time series cardiac data of the first set of the beats in a first window.

2. The method of claim 1, further comprising:
determining that respective relative timing values for a second set of the beats are within a second range of prematureness that is different than the first range; and
displaying time series cardiac data of the second set of the beats in a second window adjacent the first window.

3. The method of claim 2, wherein the first set of beats and the second set of beats share a common beat classification.

4. The method of claim 3, wherein at least some the first set of beats and some of the second set of beats are associated with a common rhythm classification.

5. The method of claim 4, wherein at least some others of the first set of beats and some others of the second set of beats are associated with a different rhythm classification.

6. The method of claim 2, further comprising:
displaying time series cardiac data of a third set of the beats in a third window adjacent the second window, wherein the third set of the beats is associated with relative timing beats equal to or greater than an average heart rate of the beats associated with the rhythm classification.

7. The method of claim 1, wherein the first range is based, at least in part, on a percentage difference below an average heart rate of the beats associated with the rhythm classification.

8. The method of claim 1, wherein the respective relative timing values are based, at least in part, on respective RR intervals associated with the beats.

9. The method of claim 8, wherein the relative timing value for each of the beats is based on dividing the RR interval with the average heart rate of the beats associated with the rhythm classification.

10. The method of claim 1, further comprising:
modifying, using the user interface, the first range by changing a minimum percentage or maximum percentage of the first range; and
updating the time series cardiac data displayed in the first window.

11. The method of claim 10, wherein the minimum percentage or the maximum percentage are modified using a drop down menu or a textbox displayed on the user interface.

12. The method of claim 1, wherein the time series data of the beats displayed in the first window is aligned with the beat’s respective R-wave peaks.

13. The method of claim 1, further comprising:
selecting one of the beat’s time series data displayed in the first window; and
displaying, in another window, a strip of time series data occurring before and after the selected beat.

14. The method of claim 1, further comprising:
modifying, using the user interface, a beat classification of the first set of the beats from a first beat classification to a second beat classification.

15. The method of claim 14, in response to the modifying, changing a rhythm classification of at least one cardiac event from a first rhythm classification to a second rhythm classification.

16. A system comprising:
a remote computing system comprising: a user interface, one or more processors, and a computer-readable medium having a set of computer-executable instructions configured to be executed by the one or more processors to cause the remote computing system to:
calculate a relative timing value for respective beats associated with a rhythm classification;
determine that respective relative timing values for a first set of the beats are within a first range of prematureness; and
display, in the user interface, time series cardiac data of the first set of the beats in a first window.

17. The system of claim 16, wherein the first range is based, at least in part, on a percentage difference below an average heart rate of the beats associated with the rhythm classification.

18. The system of claim 16, wherein the respective relative timing values are based, at least in part, on respective RR intervals associated with the beats.

19. The system of claim 18, wherein the relative timing value for each of the beats is based on dividing the RR interval with the average heart rate of the beats associated with the rhythm classification.

20. The system of claim 16, wherein the time series data of the beats displayed in the first window is aligned with the beat’s respective R-wave peaks.
